(19) United States
(12) Patent Application Publication
(10) Pub. No.: US 2024/0389943 A1
(43) Pub. Date: Nov. 28, 2024

(54) BODY FLUID ANALYTE DETECTION DEVICE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/576,370

(22) PCT Filed: Aug. 6, 2021
(86) PCT No.: PCT/CN2021/111219

§ 371 Date: Jan. 4, 2024

(51) Int. Cl. A61B 5/00; A61B 5/145

(52) U.S. Cl. CPC A61B 5/6833; A61B 5/145; A61B 5/68335

(57) ABSTRACT

A body fluid analyte detection device includes an analyte detection module for detecting analyte parameter information, and a pasting module. The pasting module includes a tape and a protective film, the first side of the tape is fixedly connected with the analyte detection module, and the second side of the tape is coated with paste material. The protective film is fixed around the outer edge of the first side of the tape, and the outer edge profile of the protective film is compatible with the outer edge profile of the tape, and the rockwell hardness of the protective film is greater than the tape. The protective film can prevent the edge of the tape from warping, avoid displacement or shedding of detection devices when in use, ensure the normal use of the detection device, enhance the user's experience.

### TECHNICAL FIELD

The present invention mainly relates to the field of medical device, and in particular, to a body fluid analyte detection device.

### BACKGROUND

The pancreas in a normal person can automatically monitor the amount of glucose in the blood and automatically secrete the required dosage of insulin/glucagon. However, for diabetic patients, the function of the pancreas is abnormal, and the pancreas cannot normally secrete required dosage of insulin. Therefore, diabetes is a metabolic disease caused by abnormal pancreatic function and also a lifelong disease. At present, medical technology cannot cure diabetes, but can only control the onset and development of diabetes and its complications by stabilizing blood glucose.

Patients with diabetes need to check their blood glucose before injecting insulin into the body. At present, most of the detection methods can continuously detect blood glucose, and send the blood glucose data to the remote device in real time for the user to view. This detection method is called Continuous Glucose Monitoring (CGM), which requires the detection device to be attached to the surface of the patients' skin, and the sensor carried by the device is inserted into the subcutaneous tissue fluid for testing.

Before the detection device is attached to the host skin surface, the static electricity on the skin surface will cause the outer edge of the tape to curl, or after the detection device is pasted to the skin surface of the host, the outer edge of the tape will become warped during the use process because of skin movement and other reasons, with the increase of the use time, the area of the curling or warping edge increases, which eventually leads to the displacement or shedding of the detection device, which affects the normal use of the detection device.

Therefore, there is an urgent need for a body fluid analyte detection device to prevent taping from warping.

### BRIEF SUMMARY OF THE INVENTION

The embodiment of the invention discloses a body fluid analyte detection device. The detection device is fixed on the skin surface of the host through adhesive tape, and a protective film is arranged on the outer edge of the other side opposite to the adhesive surface. The rockwell hardness of the protective film is higher than that of the adhesive tape, which can prevent the tape edge from curling or warping, thus preventing the detection device from displacement or falling off, and enhancing the user experience.

The invention discloses a body fluid analyte detection device, which comprises an analyte detection module for detecting analyte parameter information: The pasting module comprises a tape and a protective film, the first side of the tape is fixedly connected with the analyte detection module, and the second side opposite the first side of the tape is coated with paste material: The protective film is fixed around the outer edge of the first side of the tape, and the outer edge of the outline of the protective film is compatible with the outer edge of the tape, and the rockwell hardness of the protective film is higher than that of the tape.

According to one aspect of the invention, the rockwell hardness of the protective film is 80˜100 HRM.

According to one aspect of the invention, the tape is one of polyethylene, polypropylene, non-woven cloth or pure cotton.

According to one aspect of the invention, the thickness of the tape is 0.001˜1 μm and the thickness of the protective film is 0.01˜100 μm.

According to one aspect of the invention, the protective film is one of polycarbonate, polyamide, polyformaldehyde, polyphenyl ether, polyester, polyphenylene sulfide or polyaryl ester.

According to one aspect of the invention, the protective film has a ring structure.

According to one aspect of the invention, the analyte detection module comprises a transmitter unit, a sensor unit, a power supply and a bottom case, and the sensor unit comprises an internal part and an external part.

According to one aspect of the invention, the tape is provided with a first through hole and the internal part goes through the first through hole.

According to one aspect of the invention, the second side of the tape is covered with at least one layer of release paper before the paste module is pasted to the surface of the host skin.

According to one aspect of the invention, the release paper is provided with a second through hole, the position of the second through hole corresponds to the first through hole, so that the internal part passes through the second through hole.

Compared with the prior art, the technical solution of the present invention has the following advantages:

The invention discloses a body fluid analyte detection device, the analyte detection module is fixed to the host skin surface by a paste module, paste module includes tape and protective film, the first side of the tape fixed connection with the analyte detection module, the second side of the tape is coated with a paste material that fixes the analyte detection module to the host skin surface. The outer edge of the first side of the tape is provided with a protective film, and the rockwell hardness of the protective film is higher than that of the tape. The outer edge profile of the protective film is compatible with the outer edge profile of the tape, which can prevent the outer edge of the tape from warps, resulting in the displacement or falling off of the analyte detection module, and enhance the user experience.

Furthermore, the annular structure of the protective film can save the material used for the protective film and make the surface of the tape more beautiful.

Further, the second side of the tape is covered with at least one layer of release paper. The release paper can protect the paste material from contamination and prevent the tape from sticking to irrelevant objects.

Further, the tape is provided with a first through hole, the release paper is provided with a second through hole, the first through hole and the second hole corresponding to the position, so that the internal part of the sensor can pass through the first through hole and the second through hole in turn.

### BRIEF DESCRIPTION OF THE DRAWINGS

FIG. 1 is a schematic diagram of a body fluid analyte detection device according to an embodiment of the present invention;

FIG. 2 is a schematic diagram of the analyte detection module according to an embodiment of the invention:

FIG. 3 is a schematic diagram of the pasting module structure according to the embodiment of the invention:

FIG. 4A is the structure diagram of each layer of the paste module according to the embodiment of the invention:

FIG. 4b is a schematic diagram of the stacking sequence of pasting modules according to an embodiment of the invention:

FIG. 5 is a top view of the bottom case according to an embodiment of the present invention:

FIG. 6a-FIG. 6b are schematic diagrams of the bottom board or the side wall of the bottom case before and after the failure according to an embodiment of the present invention:

FIG. 6c-FIG. 6d are schematic diagrams of the second fastener part on the bottom case before and after the failure according to an embodiment of the present invention;

### DETAILED DESCRIPTION

As mentioned above, The prior art detection device is attached to the host skin surface, because of electrostatic or skin movement, in use process, the outer edge of the tape on the detection device will be warped, and the area of the warped edge will increase with the increase of the use time, finally, the detection device will be displaced or shedding, affecting the normal use of the detection device, and the user experience is poor.

In order to solve this problem, the invention provides a body fluid analyte detection device. A protective film is arranged on the outer edge of the first side of the tape, and the rockwell hardness of the protective film is higher than that of the tape to prevent the outer edge of the tape from becoming curved, avoid the displacement or falling off of the analyte detection device, and enhance the user experience.

Various exemplary embodiments of the present invention will now be described in detail with reference to the drawings. The relative arrangement of the components and the steps, numerical expressions and numerical values set forth in the embodiments are not to be construed as limiting the scope of the invention.

In addition, it should be understood that, for ease of description, the dimensions of the various components shown in the figures are not necessarily drawn in the actual scale relationship, for example, the thickness, the width, the length or the distance of certain units may be exaggerated relative to other structures.

The following description of the exemplary embodiments is merely illustrative, and is not intended to be in any way limiting the invention and its application or use. The techniques, methods and devices that are known to those of ordinary skill in the art may not be discussed in detail, but such techniques, methods and devices should be considered as part of the specification.

It should be noted that similar reference numerals and letters indicate similar items in the following figures. Therefore, once an item is defined or illustrated in a drawing, it will not be discussed further in following description of the drawings.

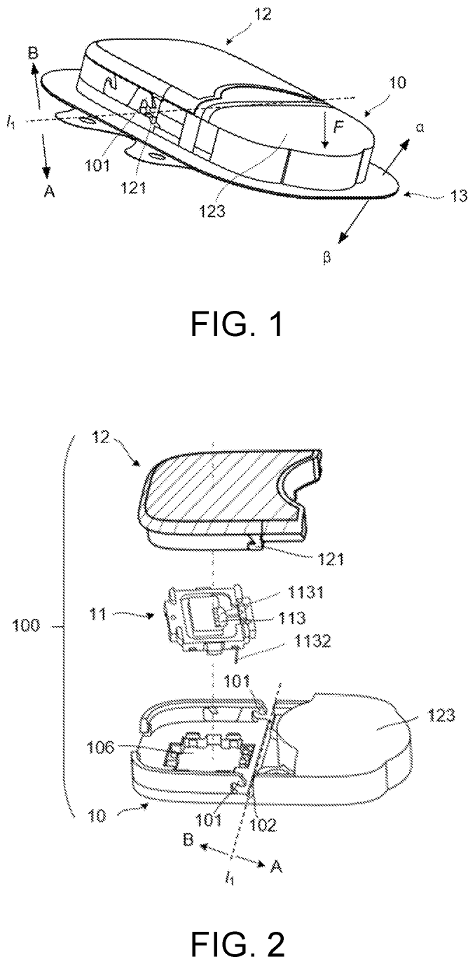

FIG. 1 is a three-dimensional structure diagram of the body fluid analyte detection device in an embodiment of the invention. FIG. 2 is a schematic diagram of analyte detection module 100 in an embodiment of the invention. FIG. 3 is a structural schematic diagram of paste module 13 in an embodiment of the invention.

The analyte detection device includes the analyte detection module 100 and the paste module 13. The analyte detection module 100 includes the bottom case 10, the transmitter unit 12, the sensor unit 11 and the battery (not shown in the figure). The bottom case 10 is used to assemble the transmitter unit 12 and the sensor unit 11. Sensor unit 11 comprises an internal part 1131 and an external part 1132, the external part 1132 is bent relative to the internal part 1131. Paste module 13 comprises tape 131, the first side a of tape 131 is fixedly connected to the bottom case 10, and the second side B is coated with paste material for pasting the analyte detection device to the host skin surface.

The material of tape 131 is one of polyethylene, polypropylene, non-woven or cotton. The tape 131 is in direct contact with the skin of the host. According to the actual use environment, the above materials can avoid the adverse reactions caused by the tape 131 contacting with the skin for a long time. In order to adapt to the host skin movement, such as tortuous, stretching, etc., the thickness of tape 131 is very thin, for example, about 0.001˜1 μm. Thin tape can cause other problems, such as static electricity from the host skin, or intense skin movement, which can cause the edges of the tape to warp. Once the edge of the tape warps, the area of the warped edge of the tape 131 will gradually increase with the increase of the use time, resulting in the decrease of the adhesion force between the tape 131 and the skin, which may lead to the displacement or shedding of the analyte detection device, affecting the user experience.

A protective film 132 is added on the outer edge of the first a face of the tape 131, and the rockwell hardness of the protective film 132 is greater than that of the tape 131. In the optimal embodiment of the invention, the rockwell hardness of the protective film 132 is 80˜100 HRM.

In the preferred embodiment of the invention, the protective film 132 is one of polycarbonate, polyamide, polyformaldehyde, polyphenyl ether, polyester, polyphenylene sulfide and polyaryl ester.

In a more preferred embodiment of the invention, the protective film 132 is polyethylene terephthalate (PET), and its rockwell hardness is 90˜95 HRM.

The outer edge profile of the protective film 132 is compatible with the outer edge profile of the tape 131. Here, the outer edge profile size, bending radius, shape and other parameters of the protective film 132 are consistent with the outer edge profile parameters of the tape 131, so that every part of the outer edge of the tape 131 can be fitted with the protective film 132.

In the preferred embodiment of the invention, the thickness of the protective film is 0.01˜100 μm.

In the preferred embodiment of the invention, the protective film 132 is annular structure, the hollow structure of the annular structure allows the protective film to be attached to the first side a of the tape 131 without interfering with the analyte detection module 100. Secondly, the inner edge of the protective film of the ring structure is consistent with the outer edge contour, which is more beautiful and enhances the user experience.

In the embodiment of the invention, the tape 131 is provided with a first through hole 1311, and the position of the first through hole 1311 corresponds to the internal part 1132 of the sensor, which is used to penetrate the internal part 1132 of the sensor to pierce the skin of the host.

The second side B of the tape 131 is also provided with at least one layer of release paper 133, the release paper 133 can prevent the adhesive material of the second side B of the tape 131 from adhering, and can protect the adhesive material from contamination of the anti-sticking paper.

In the optimal embodiment of the invention, the release paper 133 is a single silicon release paper, and its peeling force is 30˜50 g.

In the embodiment of the invention, the release paper 133 is provided with a second through hole 1331, and the position of the second through hole 1331 corresponds to the first through hole 1311, so that the internal part 1132 of sensor can pass through the first through hole 1311 and the second through hole 1331 in order to pierce the host skin.

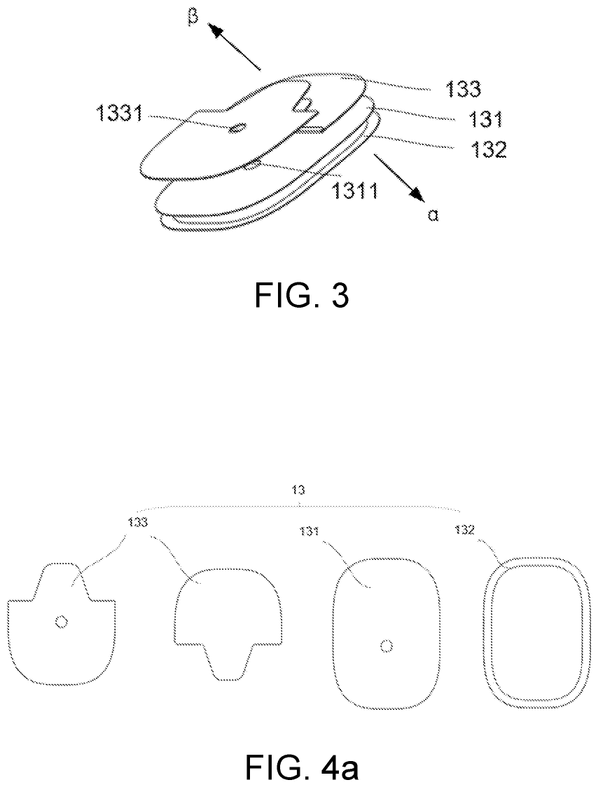

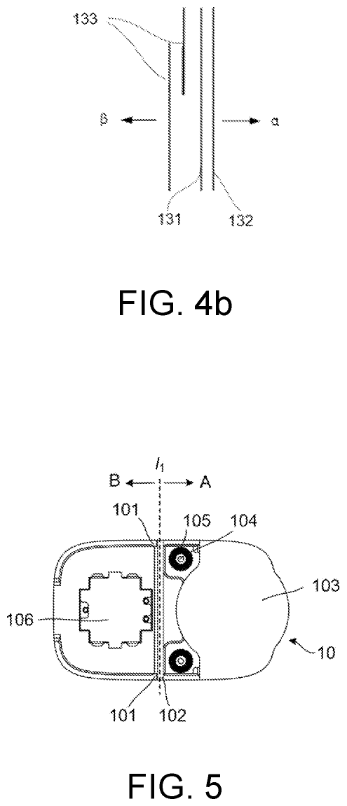

FIG. 4A is the structure diagram of each layer of sticking module 13, and FIG. 4b is the cascading sequence diagram of sticking module 13.

For the convenience of users to peel off paper 133 and tape 131, and save space, the release paper 133 is preferred to be two layers, and the peel openings are relative to each other, and the peel openings of one layer are bent outwardly and covered by the peel openings of the other layer. Combined with the above mentioned, pasting module 13 from side a to B is protective film 132, tape 131 and release paper 133 in sequence.

Continue with FIG. 1 and FIG. 2.

The detection device includes a bottom case 10, a sensor 113, and a transmitter 12. The bottom case 10, which is used to assemble the transmitter 12 and the sensor 113, and to paste the detection device on the skin surface, includes at least one fixed portion and at least one force-receiving portion. At least one second fastener part 101 is provided on the bottom case 10. The second fastener part 101 is used to fasten the transmitter 12. Preferably, in the embodiment of the present invention, the number of the second fastener part 101 is two. The two second fastener parts 101 are correspondingly provided on the side wall of the bottom case 10.

Here, the fixed portion and the force-receiving portion are relative concepts. According to the structural design of the bottom case 10 and the transmitter 12, there can be different options for the positions of the fixed portion and the force-receiving portion, which will be described in detail below.

The sensor, assembled on the bottom case 10, is used to detect the analyte parameter information of the body fluid.

The transmitter 12 is used to receive the detection signal generated by the sensor and wirelessly transmit the signal to a remote device. Therefore, the transmitter 12 is connected to the sensor.

The transmitter 12 is provided with at least one first fastener part 121 which corresponds to the second fastener part 101. With the second fastener part 101 and the first fastener part 121 fastened with each other, the transmitter 12 is assembled on the bottom case 10. Obviously, in the embodiment of the present invention, the transmitter 12 is provided with two first fastener parts 121, that is, two pairs of the first fastener part 121 and the second fastener part 101 are fastened with each other.

Here, the correspondence between the first fastener part 121 and the second fastener part 101 means that the numbers of the two are equal and their positions are corresponding.

When the bottom case 10 and the transmitter 12 are being separated, the fixed portion is fixed by a finger or other structures while the force-receiving portion receives a force in only one direction from another finger or other auxiliary structures, the bottom case 10 fails, and the second fastener part 101 and the first fastener part 121 are separated from each other, thereby separating the transmitter 12 from the bottom case 10. That means only one finger is used to apply a force in only direction to the force-receiving portion when user separates the transmitter 12 from the bottom case 10, reducing the number of the steps required. After separation, the transmitter 12 can be reused, which reduces the cost.

It should be noted here that failure is a conventional concept in the field of engineering materials. After failure, the material loses its original function and the failed part cannot be recovered again. Since the second fastener part 101 is a part of the bottom case 10, the failure of the bottom case 10 includes the failure of its bottom board or the side wall, or the second fastener part 101. Therefore, the failure modes of the bottom case 10) include one or more combinations of the fracture of the bottom board or side wall of the bottom case 10 (as shown in FIG. 4b), breakage of the bottom case 10, breakage of the second fastener part 101 (as shown in FIG. 4d), or the plastic deformation of the bottom case 10. Obviously, after the bottom case 10 fails, the bottom case 10 loses the function of fastening the transmitter 12.

The method of fixing the fixed portion includes clamping, supporting, etc., and is not specifically limited herein, as long as the condition for fixing the fixed portion can be satisfied.

Preferably, in the embodiment of the present invention, the connection line l₁ between the two second fastener parts 101 divides the bottom case 10 into side A and side B. The force-receiving portion is provided on side A while the fixed portion is on side B.

In the embodiment of the present invention, the process of separating the bottom case 10 and the transmitter 12 is as follows. Fix the fixed portion on side B with a finger, and apply a force F to the force-receiving portion in one direction with another finger, making the second fastener part 101 fail, and separating the second fastener part 101 and the first fastener part 121, thus, separating the transmitter 12 from the bottom case 10.

It should be noted that the embodiments of the present invention do not limit the positions of the second fastener part 101. For example, the two second fastener part 101 may also be provided on the bottom board of the bottom case 10, which is not specifically limited herein.

This embodiment of the present invention does not specifically limit the shape of the top view of the detection device, and the shape may also be a rounded-rectangle, rectangle, a circle, an ellipse, or other shapes.

FIG. 2 is a schematic diagram of the assembly structure of the transmitter 12 and the bottom case 10 according to the embodiment of the invention.

In this embodiment of the present invention, the detection device also includes a battery (not shown) which is used to power the transmitter and disposed in the bottom case. The part holding the battery is the battery portion 123. Preferably, in the embodiment of the present invention, the top surfaces of the battery portion 123 and the transmitter 12 are flush, which reduces the thickness of the detection device.

The battery portion 123 can be directly used as the force-receiving portion. Therefore, the battery portion 123 is set on side A of l₁. Since the battery portion 123 is thicker and larger, it is easier for the user to apply a force on the battery portion 123 and optimize the user's action.

FIG. 3 is a top view of the bottom case 10 according to this embodiment of the present invention.

Since the transmitter 12 should be charged by the battery; in the embodiment of the present invention, the bottom case 10 is provided with at least one connection hole 104 through which the transmitter 12 is electrically connected to the two electrodes of the battery.

In some embodiments of this invention, pins, disposed in the connection hole 104, are used to electrically connect to the transmitter 12. Or protruding electrical connection ends, disposed on the transmitter 12, is provided to insert into the connection hole 104. Or some leads are set through the connection hole 104. Or the wires are coated on the surface of the bottom case 10. The battery and the transmitter 12 can be electrically connected in the above connection method.

It should be noted that in other embodiments of the present invention, the connection hole 104 may not be provided. The transmitter 12 is electrically connected with the battery just only with the wires coated on the bottom case 10, which is not specifically limited herein.

Preferably, in the embodiment of the present invention, in order to seal the electrical connection position, a scaling ring 105 is provided around the connection hole 104.

Generally, the elastic scaling material can perform sealing function after being pressed. In the embodiment of the present invention, the squeezed sealing ring 105 exerts a certain clastic force on the transmitter 12. When a force F is applied to the force-receiving portion, the sealing ring 105 provides an clastic force that facilitates the separation of the transmitter 12 from the bottom case 10.

Preferably, in the embodiment of the present invention, in order to seal the connection position of the transmitter 12 and the sensor, a scale member (not shown) is provided around the sensor. Similar to the scaling ring 105 described above, when the force F is applied to the force-receiving portion, the seal member also provides an elastic force that facilitates the separation of the transmitter 12 from the bottom case 10.

It should be noted that, since the scaling performance of the electrical connection position between the transmitter 12 and the battery or the connection position between the transmitter 12 and the sensor is relatively good, other embodiments of the present invention may not provide the sealing ring 105 or the seal member.

In the embodiment of the present invention, a crease groove 102 is further provided on the bottom case 10 at a position corresponding to the connection line l₁. Since the crease groove 102 can reduce the thickness of the bottom case where it is located, the bottom case 10 is more likely to fail along the crease groove 102, making the separation easier. Obviously; the crease groove 102 can extend across the bottom board of the bottom case 10 along the connection line l₁.

Preferably, in the embodiment of the present invention, the bottom board of the bottom case 10 is further provided with a window 106 where a sensor unit 11 is assembled. The edge contour shape of the sensor unit 11 matches that of the window 106. Here, the match of the edge contour shapes of the two means that the edges of the two can be embedded into each other.

Part of the sensor 113 is provided on the sensor unit 11 while the other part is subcutaneously. The sensor 113 can be assembled on the bottom case 10 with the support of the sensor unit 11. Preferably, in the embodiment of the present invention, the sensor 113, assembled on the sensor unit 11, does not change its shape before and after assembly, that is, the sensor 113 and the sensor unit 11 are assembled on the bottom case 10 together. Therefore, after the sensor 113 is assembled, the sensor unit 11 becomes part of the bottom case 10.

It should be noted that, in the other embodiments of the present invention, the detection device can be attached to the skin directly in a whole without using the sensor unit 11 to support the sensor 113, which is not specifically limited herein.

FIG. 4a-FIG. 4b are schematic diagrams of the bottom board or the side wall of the bottom case 10, before and after the failure. FIG. 4c-FIG. 4d are schematic diagrams of the second fastener part 101 before and after the failure.

As described above, the first fastener part 121 and the second fastener part 101 are all hooks. When the force F is applied to the force-receiving portion in one direction, the bottom case 10 is fractured along the crease groove 102 as depicted by the dotted circle C. In FIG. 4c-FIG. 4d, when the force F is applied to the force-receiving portion in one direction, the second fastener part 101 is fractured.

In other embodiments of the present invention, the first fastener part 121 and the second fastener part 101 may be a locking hole and a locking block, or a locking block and a locking slot, respectively, which is not specifically limited herein.

In other embodiments of the present invention, the portion of the bottom case 10 where the transmitter 12 is assembled can also be a force-receiving portion, which is not specifically limited herein. It should be pointed out here that although the force F may be directly applied on the transmitter 12, the actual effect of the force F is only displayed on the bottom case 10, that is, the force F only changes the condition of the bottom case 10 (such as changes in shape and structure etc.), not that of the transmitter 12, and at this time, it can be regarded that the force is applied on the force-receiving portion of the bottom case 10 by the user.

### The Second Embodiment

Compared with the first embodiment, the second embodiment is different in that only one first fastener part is provided on the transmitter, only one second fastener part is correspondingly provided on the bottom case, and the shape of the top view of the detection device is ellipse.

FIG. 5 is a schematic diagram of a detection device including only one first fastener part and one second fastener part according to this embodiment of the present invention.

One first fastener part and one second fastener part are fastened with each other at position D, that is, there is only a pair of the first fastener part and the second fastener part that fasten with each other.

Preferably, in the embodiment of the present invention, the side wall of the bottom case 20 is provided with a convex portion 211. The convex portion 211 is a force-receiving portion, while the rest part of the bottom case 20 is the fixed portion. The convex portion 211 is easy to apply the force F, which is convenient for the user to separate.

When a force is applied to the convex portion 211, the bottom case 20 fails, and the first fastener part and the second fastener part are separated from each other, thus, making the transmitter 22 and the bottom case 20) separate.

For the failure mode of the bottom case 20, the type and the fastening mode of the first fastener part and the second fastener part, please refer to the foregoing description, which will not be repeated herein.

### The Third Embodiment

Compared with the aforementioned embodiments, the fourth embodiment is different in that the detection device includes a plurality of force-receiving portions.

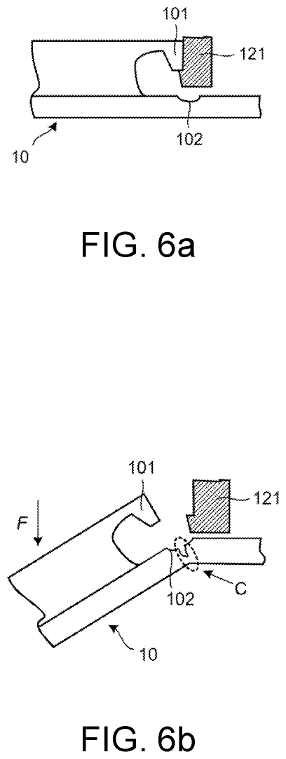

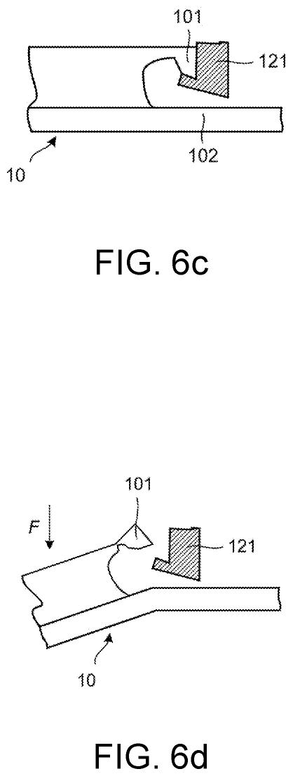

FIG. 6 is a top view of a detection device including two force-receiving portions in one detection device according to an embodiment of the present invention.

In the embodiment of the present invention, the transmitter 32 is provided with four first fastener parts while the bottom case 30 is correspondingly provided with four second fastener parts. The corresponding first fastener parts and second fastener parts are fastened with each other at D₁-D₁′ and D₂-D₂′, that is, there are four pairs of mutually fastened first fastener parts and second fastener parts. On the bottom case 30, the connection lines of D₁-D₁′ and D₂-D₂′ are l₂ and l₃, respectively. Side A and side B of l₂ and l₃ are provided with the force-receiving portion and the fixed portion, respectively. Therefore, l₂ and l₃ divide the bottom case 30 into three parts: the force-receiving portion, the fixed portion and the force-receiving portion. In the embodiment of the present invention, two force-receiving portions are provided on both sides of the bottom case 30, and the one fixed portion is disposed between the two force-receiving portions.

When the transmitter 32 and the bottom case 30 are being separated, two thumbs are supported under the fixed portion to fix it while the two index fingers respectively apply a force to the force-receiving portion in one direction on both sides of the detection device, making the bottom case 30 fail easily.

For the failure modes of the bottom case 30, the types and the fastening modes of the first fastener part and the second fastener part, please refer to the foregoing description, which will not be repeated here.

It should be noted that, in other embodiments of the present invention, more first fastener parts and second fastener parts may be provided. Among them, the failure of at least one pair of the first fastener part and the second fastener part (similar to the manner shown in FIG. 5), or the failure of the two corresponding pairs of the first fastener parts and the second fastener parts (similar to the manner shown in FIG. 2), or the failure of multiple pairs of corresponding first fastener parts and second fastener parts (similar to the method shown in FIG. 6) can all separate the bottom case and the transmitter.

In summary; the present invention discloses a body fluid analyte detection device. The bottom case includes at least one fixed portion and at least one force-receiving portion, and the battery is disposed in the bottom case. The force is applied to the force-receiving portion in only one direction to make the bottom case fail and thereby separate the transmitter and the bottom case, which makes the transmitter reused, reducing the cost, simplifying user action, and enhancing user experience.

While the invention has been described in detail with reference to the specific embodiments of the present invention, it should be understood that it will be appreciated by those skilled in the art that the above embodiments may be modified without departing from the scope and spirit of the invention. The scope of the invention is defined by the appended claims.

1. A body fluid analyte detection device, comprising:
an analyte detection module configured to detect analyte parameter information;
a pasting module, comprising a tape and a protective film, wherein a first side of the tape is fixedly connected with the analyte detection module, and a second side of the tape opposite to the first side is coated with a paste material, the protective film is fixed around an outer edge of the first side of the tape, an outer edge of the protective film is conformal to the outer edge of the first side of the tape, and a rockwell hardness of the protective film is greater than a rockwell hardness of the tape.

2. The body fluid analyte detection device of claim 1, wherein the rockwell hardness of the protective film is 80˜100 HRM.

3. The body fluid analyte detection device of claim 1, wherein the tape is one of polyethylene, polypropylene, non-woven cloth or pure cotton.

4. The body fluid analyte detection device of claim 1, wherein a thickness of the tape is 0.001˜1 μm, and a thickness of the protective film is 0.01˜100 μm.

5. The body fluid analyte detection device of claim 1, wherein the protective film is one of polycarbonate, polyamide, polyformaldehyde, polyphenyl ether, polyester, polyphenylene sulfide or polyaryl ester.

6. The body fluid analyte detection device of claim 1, wherein the protective film has a ring structure.

7. The body fluid analyte detection device of claim 1, wherein the analyte detection module comprises a transmitter unit, a sensor unit, a power supply and a bottom case, and the sensor unit comprises an internal part and an external part.

8. The body fluid analyte detection device of claim 7, wherein the tape is provided with a first through hole, and the internal part passes through the first through hole.

9. The body fluid analyte detection device of claim 8, wherein before the paste module is pasted to the host skin a surface, the second side surface of the tape is covered with at least one layer of a release paper.

10. The body fluid analyte detection device of claim 9, wherein the release paper is provided with a second through hole, and a position of the second through hole corresponds to the first through hole, so that the internal part passes through the second through hole.
